(19) United States
(12) Patent Application Publication
(10) Pub. No.: US 2010/0198299 A1
(43) Pub. Date: Aug. 5, 2010

(54) BIOLOGICAL IMPLANTABLE FUNCTIONAL DEVICE AND VISION REGENERATION ASSISTING APPARATUS

(75) Inventors: Kenzo Shodo, Kyoto-shi (JP); Yasuo Terasawa, Ohbu-shi (JP)

Correspondence Address: SUGHRUE-265550, 2100 PENNSYLVANIA AVE. NW, WASHINGTON, DC, 20037-3213, US

(73) Assignee: NIDEK CO., LTD., Aichi (JP)

(21) Appl. No.: 12/697,633

(22) Filed: Feb. 1, 2010

(30) Foreign Application Priority Data
Feb. 2, 2009 (JP) 2009-021914

(51) Int. Cl. A61F 9/08; A61N 1/36

(52) U.S. Cl. 607/53

(57) ABSTRACT

A biological implantable functional device comprises: a casing having a space for accommodating an electronic device and formed with an opening; a bendable flexible wiring substrate in which a wiring is formed in a predetermined pattern so as to correspond to an device-side terminal of the electronic device; a casing inner connecting terminal to be connected to an electric substrate provided outside the casing; and a bump to be electrically connected with the flexible wiring substrate and the casing inner connecting terminal; and a cover for sealing the opening of the casing to hermetically seal the electronic device.

### BACKGROUND

The present invention relates to a biological implantable functional device in which an electronic device operated in a body is hermetically sealed. Also, the invention relates to a vision regeneration assisting apparatus which regenerates a patient's vision by implanting a hermetically sealed functional device into a patient.

In the past, there were known various devices used for treating a disease, substituting a function, or obtaining body information by implanting a precise device having a semiconductor integrated circuit into a body. When an electronic circuit (electronic device) such as an integrated circuit as the functional device implanted into the body comes into direct contact with living tissue, body fluids invade the circuit, so that the function of the circuit is degraded. For this reason, various methods have been studied so as to protect the electronic circuit from the body fluid or the like. In the field of a semiconductors, there is known a technology which inserts an electronic circuit into a casing formed from ceramics so as to protect the electronic circuit from the influence of moisture or the like, disposes an input/output terminal or the like connected to the circuit on the outside, and seals (hermetically seals) the casing (for example, refer to US2004207485). A method is supposed in which the implanted electronic circuit does not come into direct contact with living tissue through the application of such a technology.

In recent years, as one blindness treatment method, a vision regeneration assisting apparatus has been examined which implants a device having a plurality of electrodes into an eye or the like, and stimulates cells constituting the vision by outputting a pulsar stimulus current thereto from the electrode so as to substitute a part of lost visual function (for example, refer to US2003192784). Such a vision regeneration assisting apparatus includes an internal body device which is disposed inside the eye, and the internal body device is provided with an electrode used for giving an electric stimulus to cells constituting the retina and a control unit including an integrated circuit used for controlling the electrode.

The device implanted into a living body, and particularly, a portion having limited space such as an eyeball or the head is required to be as small as possible. However, in the technology disclosed in JP2004207485, since wire joining is used upon electrically connecting an connecting terminal of the electronic circuit accommodated in the casing to an connecting terminal drawn out from the casing, it is necessary to ensure a predetermined space inside the casing. As a result, it is difficult to decrease the size of the casing.

### SUMMARY

The present invention is contrived in consideration of the above-described problems, and a technical object of the invention is to provide a biological implantable functional device in which whole size is minimized by suppressing a required inner space for sealing an electronic device from an exterior and a vision regeneration assisting apparatus.

In order to achieve the above-described object, the present invention provides the following arrangements.

(1) A biological implantable functional device comprising:

a casing having a space for accommodating an electronic device and formed with an opening;

a bendable flexible wiring substrate in which a wiring is formed in a predetermined pattern so as to correspond to an device-side terminal of the electronic device;

a casing inner connecting terminal to be connected to an electric substrate provided outside the casing;

a bump to be electrically connected with the flexible wiring substrate and the casing inner connecting terminal; and a cover for sealing the opening of the casing to hermetically seal the electronic device.

(2) The biological implantable functional device according to (1), wherein the hermetically sealing is performed by ultrasonic joining or pressure joining.

(3) The biological implantable functional device according to (1), wherein

the biological implantable functional device is used for a vision regeneration assisting apparatus, and

the casing inner connecting terminal is electrically connected to an exterior substrate which is covered by a biocompatible material, which is processed to have a thickness configured the exterior substrate to be bent, and includes a plurality of electrodes connected to a plurality of lead wires formed within the exterior substrate.

(4) The biological implantable functional device according to (3), wherein the casing inner connecting terminal is electrically connected to the exterior substrate through a bump.

(5) The biological implantable functional device according to (1), wherein

the flexible wiring substrate includes a base which is a film-shaped member formed from an insulating material, and

the wiring pattern is formed on a surface of the base.

### BRIEF DESCRIPTION OF THE DRAWINGS

FIG. 1 is a schematic diagram showing an external shape of a vision regeneration assisting apparatus according to an embodiment.

FIGS. 2A and 2B are diagrams showing a configuration of an internal body device of the vision regeneration assisting apparatus according to the embodiment.

FIG. 3 is a schematic diagram of a section in the vicinity of a multiplexer.

FIGS. 4A to 4D are diagrams showing a configuration of sealing the multiplexer.

FIG. 5 is a diagram showing a state where the internal body device is provided in a patient's eye.

FIG. 6 is a block diagram showing a control system of the vision regeneration assisting apparatus according to the embodiment.

### DESCRIPTION OF EXEMPLARY EMBODIMENTS

An exemplary embodiment of the invention will be described with reference to the drawings. In the embodiment, a functional device (unit) to be implanted into a body will be described as a part of an internal body device of a vision regeneration assisting apparatus mounted with an electronic device (electronic circuit). The functional device is hermetically sealed by the following configuration.

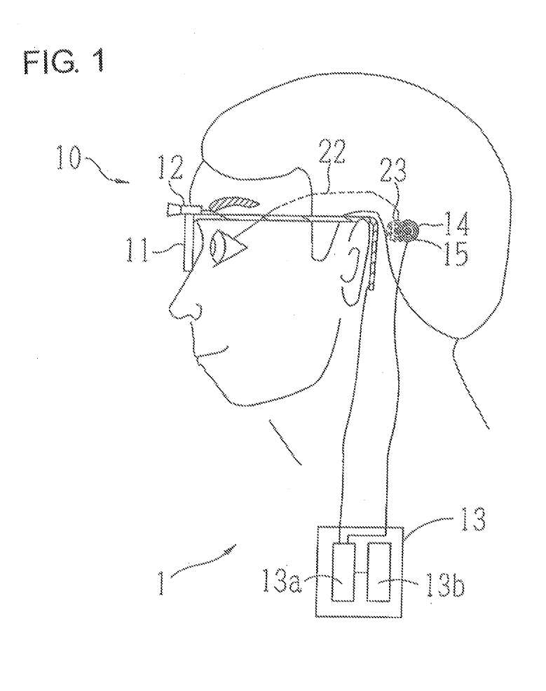

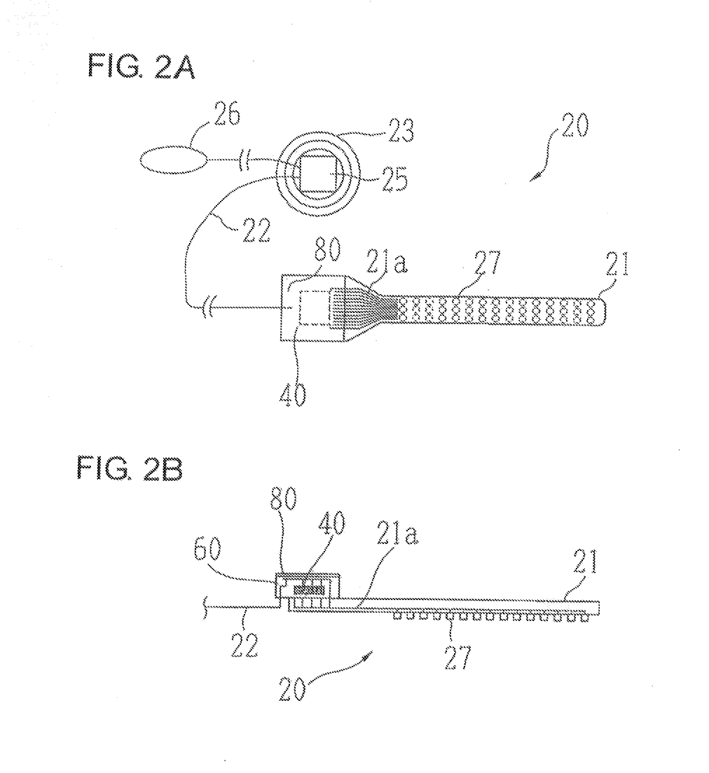

FIG. 1 is a schematic diagram showing an external shape of a vision regeneration assisting apparatus. FIG. 2 is a diagram showing an internal body device of the vision regeneration assisting apparatus used in the embodiment.

As shown in FIGS. 1 and 2, the vision regeneration assisting apparatus 1 includes an external body device 10 which photographs an objective world and an internal body device 20 which gives an electric stimulus to cells constituting a retina so as to promote the regeneration of the vision. The external body device 10 includes a visor 11 which is put on a patient, a photographing device 12 which includes a CCD camera, etc. mounted to the visor 11, an external device 13, a transmitter 14 which includes a primary coil, etc.

The external device 13 is provided with a pulse signal converter 13a which includes a calculation process circuit such as a CPU, and a battery 13b which is used to supply: power to the vision regeneration assisting apparatus 1 (the external body device 10 and the internal body device 20). The pulse signal converter 13a performs image processing on a subject image photographed by the photographing device 12 so as to convert image process data into electric stimulus pulse data used for regenerating the vision. The transmitter 14 is able to transmit (wirelessly transmit) the electric stimulus pulse data converted by the pulse signal converter 13a and power for driving the internal body device 20 to be described later as an electromagnetic wave to the internal body device 20. In addition, a magnet 15 is mounted to the center of the transmitter 14. The magnet 15 is used to improve the data transmission efficiency using the transmitter 14 and to fix the position of a receiver 23 to be described later.

The visor 11 is formed in a glasses shape, and can be used while being mounted to a position before a patient's eyeball as shown in FIG. 1. The photographing device 12 is mounted to the front surface of the visor 11 so as to photograph a subject which is seen by the patient.

The internal body device 20 shown in FIGS. 2A and 2B includes a substrate 21 which is provided with a plurality of electrodes 27, a cable 22, the receiver 23 which includes a secondary coil used for receiving the electromagnetic wave from the external body device 10, a control unit 25 which extracts the electric stimulus pulse, a multiplexer control signal (hereinafter, simply referred to as a control signal) for distributing the electric stimulus pulse to each of designated electrodes, and the power from the electromagnetic wave including the power and the electric stimulus pulse data received, by the receiver 23, a returning electrode (counter electrode) 26, a multiplexer 40 which includes an electronic device such as a semiconductor, etc. A magnet (not shown) is disposed in the receiver 23, and is used to fix the magnet 15 of the external body device 10.

The multiplexer (controller) 40 as a functional device including a semiconductor integrated circuit is provided on the substrate 21 while being electrically connected to the control unit 25 through the cable 22 and being electrically connected to each of electrodes 27 through a lead wire 21a wired on the substrate 21 so as to distribute the electric stimulus pulse (stimulus current) for stimulating cells constituting the retina to each of the electrodes 27 on the basis of the electric stimulus pulse and the control signal transmitted from the control unit 25. The multiplexer 40 is provided on the substrate 21 through a casing 60, and is also hermetically sealed (sealed) by a cover member 80.

The substrate 21 is formed in such a manner that a base portion is formed by a resin such as polyimide having high biocompatibility formed in an elongate plate shape so as to be bendable, and a plurality of lead wires 21a are wired thereon. The wiring of the substrate 21 is formed in such a manner that corrosion resistant metal material is deposited on the base portion by using known photoresist, vacuum depositing, sputtering, or the like, and a conduction layer used as the lead wires 21a shown in FIGS. 2A and 2B is formed. After forming the conduction layer, the mask is removed, and an insulation layer having a predetermined thickness is formed thereon by coating or adhering so as to coat the conduction layer. As a material used for the insulation layer, for example, an insulation material such as polyimide or poly (p-xylylene) having high biocompatibility may be used. The insulation layer at the terminal end position of the obtained lead wire 21a is perforated by a method such as RIE (reactive ion etching) so as to expose the terminal end of the lead wire 21a, and an electrode material is laminated (deposited) thereon so as to form a bump as the electrode 27. Through the above-described processes, the substrate 21 having the lead wire 21a or the electrode 27 formed thereon is manufactured. In addition, in the case where a plurality of lead wires 21a needs to be three-dimensionally disposed, it is possible to form three-dimensional wiring by performing these processes plural times. Further, the electrode and the lead wire are not limited to the above-described configuration. A configuration may be adopted in which the electric stimulus pulse signal is appropriately output. For example, an electrode is cut out from a bulk material, and a conduction wire as a lead wire is connected to the electrode so as to be connected to a connecting terminal of the multiplexer (casing). Then, a substrate used for combining the electrode and the lead wire is formed.

As shown in FIG. 2A, a plurality of electrodes 27 are disposed in a matrix shape at the same interval in the longitudinal direction of the substrate 21 or a plurality of electrodes are formed in two dimensions at the same interval so as to form an electrode array. Although the number of electrodes 27 is determined in accordance with the resolution upon regenerating the vision, the number is about several tens to several hundreds. In addition, if there is no problem in an installation space of the electrode or a wiring substrate, the number may be equal to or more than several tens to several hundreds. Further, as described above, the electrode 27 formed on the substrate 21 is a conductive material such as gold or platinum having excellent biocompatibility and corrosion resistant property, and is formed in the terminal end of each lead wire 21a formed on the substrate 21.

The control unit 25 includes a semiconductor integrated circuit (LSI) including several control circuits such as a circuit which divides the electric stimulus pulse data and the power included in the electromagnetic wave received by the receiver 23, a conversion circuit which obtains the electric stimulus pulse for obtaining a vision and the control signal of the multiplexer 40 on the basis of the electric stimulus pulse data, and an electronic circuit which transmits the converted electric stimulus pulse and the control signal to the multiplexer 40. The electric stimulus pulse data is converted by the control unit 25 having the above-described configuration, and the control signal and the electric stimulus pulse created by the conversion process are transmitted to the multiplexer. 40. The multiplexer 40 receiving the electric stimulus pulse and the control signal transmits (distributes) the electric stimulus pulse for stimulating cells constituting the retina to each of the electrodes 27 in accordance with the control signal. That is, the electric stimulus pulse is controlled by the multiplexer 40. In addition, a plurality of electrodes 27 are separately connected to the multiplexer 40 through a plurality of lead wires 21a formed on the substrate 21. Further, the multiplexer 40 is used to receive the power from the control unit 25.

The cable 22 is coated by a material (not shown) having an insulating property and high biocompatibility, and is used to electrically connect the control unit 25 to the multiplexer 40.

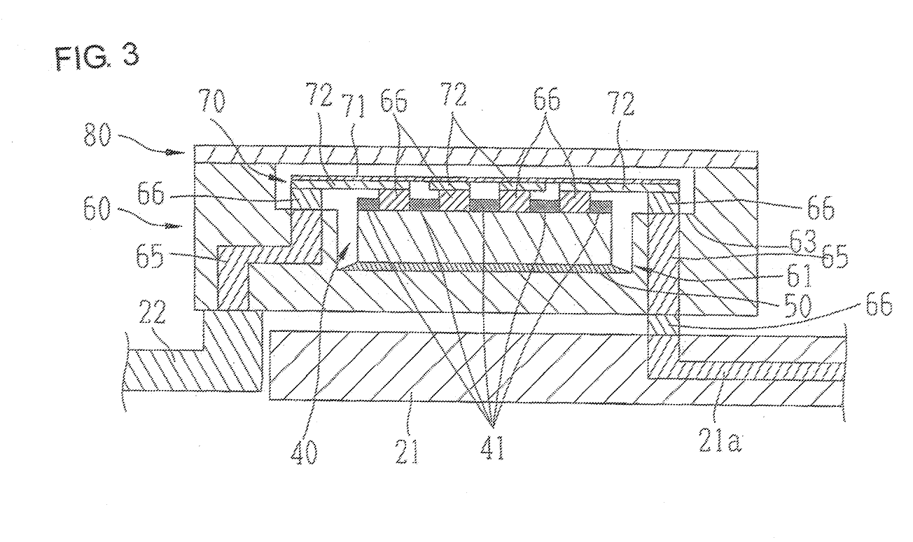

Next, a peripheral configuration of the multiplexer 40 will be described. FIG. 3 is a schematic sectional view showing an enlarged part in the vicinity of the multiplexer 40 shown in FIG. 2A. As shown in FIG. 3, the multiplexer 40 is disposed inside the casing 60 joined onto the substrate 21, and is covered by the cover member 80. In the embodiment, the multiplexer 40 includes a main body having a rectangular parallelepiped shape (plate shape), and a terminal of the wiring (inner wiring) used for allowing the integrated circuit of the multiplexer 40 to function is formed on one surface (upper surface) of the main body of the multiplexer 40 in a predetermined pattern. A bonding pad (not shown) as an input/output terminal is formed in the connecting pattern. The bonding pad is provided with a bump 66 which is a connecting terminal (device-side terminal) used to be connected to the wiring of the casing 60 to be described later. In the multiplexer 40, a surface opposite to the surface provided with the bump 66 is disposed inside the casing 60 so as to be brought into contact with the bottom of the casing 60. In addition, the surface provided with the bump 66 is covered by an insulator 41 except for the bump 66.

The casing 60 is formed from an insulating material such as ceramics, airtightness against gas and moisture (permeability is low), and biocompatibility so as to have a box shape. An opening is provided in the upper portion thereof the casing 60, a concave portion 61 is provided so as to accommodate the multiplexer 40 therein, and a stepped portion 63 is provided in the vicinity of the concave portion 61 so as to more protrude to the inside than the inner wall of the casing 60. The height position of the stepped portion 63 is formed so as to be substantially the same as that of the upper surface of the multiplexer when the multiplexer 40 is accommodated in the concave portion 61. Further, in the casing 60, a wiring (via) 65 used to be electrically connected to the connecting pattern of the multiplexer 40 is provided so as to perforate the casing 60 from the inside to the outside of the casing. Furthermore, the inside of the casing 60 indicates the side of the inner hole which is hermetically sealed. The wirings 65 are provided as many as the number corresponding to a plurality of bonding pads provided in the multiplexer 40. In addition, the casing-side terminal (the casing inner side end) of the wiring 65 is formed in the stepped portion 63 provided in the vicinity of the concave portion 61. The terminal of the wiring 65 formed in the stepped portion 63 is provided with a bump 66 which is used to be connected to other wirings.

In addition, although the wiring 65 may be formed so as to be appeared on the bottom of the casing 60 while vertically perforating the casing 60 from the terminal formed in the stepped portion 63, the wiring 65 may be formed in the casing 60 so as to be bent in order to more easily connect the wiring 65 drawn out to the outside of the casing 60 to the lead wire 21a of the substrate 21. Accordingly, since it is possible to freely lay out the position of the terminal appeared on the outside of the casing 60, it is possible to avoid the concentration of the wirings and to facilitate the connection process of the wirings. With such a configuration, the penetration distance of the wiring 65 inside the casing 60 is extended, and hence the boundary face is extended. Accordingly, invasion of a body fluid or the like hardly occurs.

The casing 60 is manufactured by the molding of fine ceramics (ceramics). In the embodiment, a manufacture procedure using alumina as the material will be described. Alumina is stretched in a plate shape, and through holes are formed at a part corresponding to the wiring 65. The holes for forming the wirings 65 are provided as many as the number of provided electrodes 27 or other electric connection purposes. A paste to be the wiring 65 is inserted (filled) in the holes so as to have no gap. The paste is obtained by mixing (or dissolving a conductor in volatile solvent) volatile liquid with a conductor having biocompatibility, for example, fine particles of metal such as platinum, gold, titanium, tungsten, or molybdenum, and the form thereof is freely determined. The alumina is deposited while arranging the form so as to form the concave portion 61 and the stepped portion 63. At this time, in the case where the wiring 65 is bent in the inside of the casing, layers having alumina and paste formed thereon to have a predetermined layout are laminated. For example, by the combination of the layout in which the paste is disposed in a direction perpendicular to the diameter of the wiring 65 and the layout in which the paste is stretched in the horizontal direction, wirings (bent wirings) are obtained which are stretched in the horizontal direction (lateral direction) with respect to the wiring disposed in a direction perpendicular to the casing.

When the wirings manufactured in this manner is heated at a high temperature in a furnace (sintering), the boundary face of the wiring portions of the casing 60 comes into close contact with each other due to the contraction of powdered particles of alumina and the expansion of metal. In addition to the ceramics, the casing having high airtightness of the wiring portion is manufactured. Further, since the wiring is manufactured by platinum as metal (conductor) having high biocompatibility, even when the wiring portion of the casing is invaded in living tissue, it is possible to reduce negative influences on living tissue.

Incidentally, in the embodiment, the casing 60 is molded by alumina, but the invention is not limited thereto. An insulating material, airtightness, and biocompatibility may be used. Fine ceramics other than alumina, for example, glass, ferrite, diamond, etc may be used. A mineral having an insulating property, airtightness, and biocompatibility, for example, sapphire (corundum) etc may be also used.

A connection substrate 70 (connecting wiring substrate) is a flexible wiring substrate in which wiring is formed in a predetermined pattern and which is bendable. The connection substrate 70 includes a film-shaped bendable base 71 which has an insulating property and has such a size that the connection substrate can be inserted into the inner hole of the casing 60, and a connecting pattern portion 72 which is formed on the upper surface (one surface) of the base 71 and is formed in a predetermined pattern so as to electrically connect the casing inner terminal portion (bump 66) of the wiring 65 to the terminal portion (bump 66) on the multiplexer 40.

The base 71 is preferably formed from a material such as a resin having biocompatibility which is bendable in a predetermined thickness. Here, a resin such as polyimide or poly (p-xylylene) having biocompatibility and an insulating property is employed for the molding. The base 71 is formed in such a size that the base covers the terminal portion (bump 66) of the wiring 65 formed in at least the stepped portion 63 and the multiplexer 40 accommodated in the casing 60. The thickness is about 10 to 100 μm.

The wiring of the connecting pattern portion 72 is preferably formed from a conductor having biocompatibility, for example, a metal such as gold, platinum, and titanium, and is formed on the base 71 in a predetermined connecting pattern through photolithography so as to correspond to the terminal formation position on the stepped portion 63 and the terminal formation position on the multiplexer 40. The connecting pattern may be formed from metal such as copper or silver. The connecting pattern portion 72 is formed to have a thickness of about 10 to 100 μm. Therefore, the entire thickness of the connection substrate 70 is about 20 to 200 μm, and is preferably about 30 to 70 μm. The connecting pattern portion 72 is formed inside the base 71, and only the connecting terminal portion may be exposed to the surface as the connecting terminal. In this case, a surface from which the connecting terminal is exposed is a wiring surface.

In the connection substrate 70, the connecting pattern portion 72 and the bump 66 are joined to each other through ultrasonic joining or pressure joining, so that the multiplexer 40 is electrically connected to the wiring 65. The bending degree of the connection substrate 70 is set to the bending degree of the connection substrate 70 in accordance with the inclination upon installing the bump 66 (multiplexer 40) in the state where the electric connection is maintained.

Since the base 71 has an insulating property, even when the cover member 80 to be described later is formed from a material having conductivity, the connection substrate 70 and the cover member 80 are not electrically connected to each other when they come into contact with each other. For this reason, it is possible to decrease the entire height of the casing 60 as low as possible.

The cover member 80 molded so as to shield the multiplexer 40 is formed from a material having high biocompatibility and airtightness, for example, metal such as ceramics, titanium, platinum, and gold so as to have a plate shape. The cover member 80 is formed to have a size of closing the opening of the casing 60.

In the case where the casing 60 is formed from ceramics and the cover member 80 is formed from metal, metallizing is performed on the connection portion between the casing 60 and the cover member 80 in order to perform a strong joining operation. The metallizing is a technology of forming a metallic layer on a surface, etc. of nonmetals such as ceramics. The metallizing is performed by direct brazing or active metal method. Here, the metal used for the metalizing is a precious metal with biocompatibility such as titanium, gold, or platinum. Since the cover member 80 is metal, it is possible to easily perform the hermetic sealing process by a technology such as seam joining. The metallizing may be performed in advance upon forming the casing 60.

In the case where the casing 60 and the cover member 80 are ceramics, the joining operation therebetween is performed by welding or brazing. In the case of brazing, joining having high biocompatibility is performed. In addition, the connection portion of ceramics may be joined by metallizing.

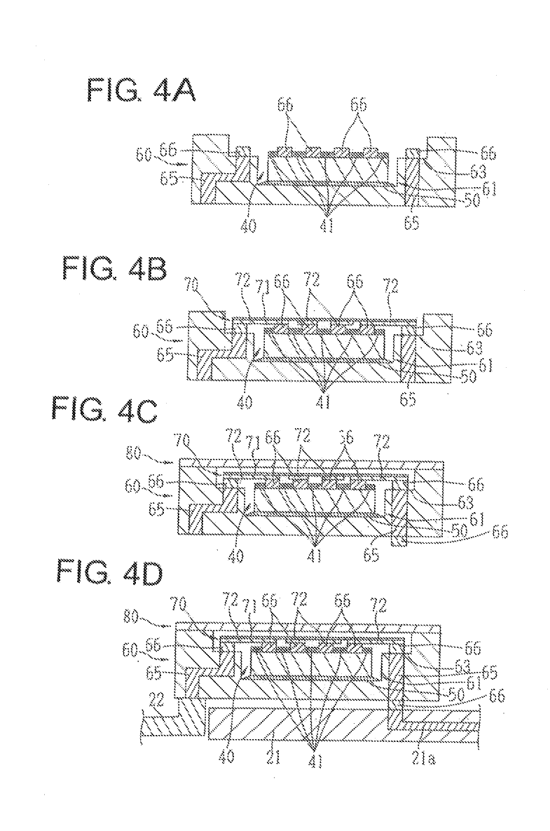

Next, a process of hermetically sealing the multiplexer 40 will be described with reference to FIG. 4B. First, as shown in FIG. 4A, the casing 60 is joined to the multiplexer 40. An adhesive 50 is applied to the bottom of the concave portion 61, and the multiplexer 40 is accommodated in the concave portion 61 so as to fix the multiplexer 40 to the casing 60. At this time, a surface provided with the bump 66 (bonding pad) in the multiplexer 40 is set to an upper surface. In addition, preferably, the adhesive 50 to be used has flexibility. Accordingly, it is possible to alleviate a shock or the like applied to the multiplexer 40. The adhesive may not be used.

Next, as shown in FIG. 4B, the connection substrate 70, the multiplexer 40, and the casing 60 are connected to each other. After performing a positioning operation between the bump 66 and the connecting pattern portion 72, ultrasonic joining or pressure joining is performed from the upside, so that the bump 66 and the connecting pattern portion 72 are joined to each other, and the multiplexer 40 and the wiring 65 are electrically connected to each other.

Since the connection substrate 70 is formed to be bendable (flexible), even when there is a slight difference in height between the bump 66 of the multiplexer 40 and the bump 66 on the stepped portion 63, it is possible to reliably connect (join) the entire area of the connection position without connection error by performing ultrasonic joining or pressure joining on the connection substrate 70.

After joining the connection substrate 70, a gap between the multiplexer 40 and the connection substrate 70 is filled with a filler (adhesive) having an insulating property. Due to the filer, the connection degree between the multiplexer 40 and the connection substrate 70 increases, and the connecting pattern, etc. on the multiplexer 40 during the connection operation are protected. Since the filler is filled into the gap, air of the connection position is excluded. A configuration may be adopted in which epoxy flows into the gap between the multiplexer 40 and the connection substrate 70 after the joining operation.

Furthermore, in the above-described process, a configuration is adopted in which the connection substrate 70 is connected after accommodating the multiplexer 40 in the casing 80, but the invention is not limited thereto. A process may be adopted in which the multiplexer 40 is accommodated in the casing 80 after joining the multiplexer 40 to the connection substrate 70, and the connecting terminal of the connection substrate 70 is connected to the bump 66 of the casing 80. In this case, since it is possible to obtain highly precise flatness between (the bump 66 of) the multiplexer 40 and the connection substrate 70, flip chip joining may be used for the connection operation therebetween. Accordingly, it is possible to easily perform the positioning operation between the connection substrate 70 and the bump 66 of the casing 80.

Next, as shown in FIG. 4C, the casing 60 is joined to the cover member 80 (which is formed from titanium in this example). This process is performed under the presence of inert gas (argon or nitrogen gas). Here, argon is used as the inert gas. Accordingly, argon is filled in the internal space of the cover member 80. Under the presence of argon, the cover member 80 and the casing 60 where the connection position between the casing 60 and the cover member 80 is subjected to metalizing in advance are positioned each other and are come into contact with each other. Subsequently, by tracing a roller (not shown) the cover member 80 while applying heat and pressure to the cover member, an area subjected to metalizing of the casing 60 and the cover member 80 are subjected to seam joining.

Since the casing 60 and the cover member 80 are joined to each other in the above manner, the multiplexer 40 is sealed so as to be protected from the outside. That is, the hermetic sealing casing of the multiplexer 40 is constituted by the casing 60 and the cover member 80. In the embodiment, the internal space of the cover member 80 is filled with an inert gas, but the invention is not limited thereto. A configuration may be adopted in which a gap is filled with a filler so as to protect the multiplexer 40. Further, the hermetic seal may be performed under the vacuum state. Further, the cable 22 and the wiring 65 of the casing 60 are joined to each other. The front end of the cable 22 is brought into contact with the wiring 65 so as to be joined to each other under a high temperature state and a high pressure state. The cable 22 and the wiring 65 formed from metals are strongly joined to each other.

Finally, as shown in FIG. 4D, the casing 60 accommodating the multiplexer 40 is joined to the substrate 21. As in the above-described process, the bump 66 provided with the external terminal of the wiring 65 perforating the inside of the casing 60 is positioned to the exposed portion of the lead wire 21a so as to come into contact with each other. Subsequently, an ultrasonic wave or the like is applied to the contact portion so as to join the casing 60 to the substrate 21. At this time, the gap between the casing 60 and the substrate 21 is filled with an epoxy resin having biocompatibility. Further, although in the embodiment, such a configuration is adopted in which the bump 66 is formed on the casing 60 used for the joining operation between the casing 60 and the substrate 21, the invention is not limited thereto. Such a configuration may be adopted in which a bump is formed on the lead wire 21a exposed to the substrate 21. In the drawing, the lead wire 21a and the wiring 65 are depicted in one position, but in fact, the lead wires 21a are formed as many as the number of electrodes, and the wirings 65 are formed so as to correspond to the lead wires 21a, where they are joined to each other at a plurality of positions.

After the above-described series of joining, the entire area of the substrate 21 except for the electrodes 27 is embedded by a resin having high biocompatibility (silicon, poly (p-xylylene), and polyimide having high biocompatibility). By the embedding operation using the resin, it is possible to further seal the multiplexer 40 so as not to contact with living tissue.

As described above, since the connection substrate 70 is used for the wiring inside the casing 60, it is possible to seal the multiplexer 40 without increasing the internal body device 20. Since the wiring 65 is provided while molding the casing 60 by an insulating material, it is possible to handle a plurality of input/output terminals drawn out from the multiplexer 40 and the wirings (and other electric connection wirings) for the electrodes 27, and to perform the sealing operation in a comparatively simple manner. In addition, since the cover member 80 is formed from metal, it is possible to manufacture the cover member by using a known technology so as to have a thickness thinner than that of the case of molding the cover member by ceramics, and thus to easily manufacture the cover member. In addition, in the multiplexer 40, since the substrate 21 and the casing 60 are joined to each other by an ultrasonic wave after the casing 60 and the cover member 80 are subjected seam joining, it is possible to reduce joining damage to the substrate 21 compared with a case where the joining operation is performed under a high humidity state and a high pressure state. In addition, in the above-described hermetic sealing method, an example of the vision regeneration assisting apparatus is described, but the invention is not limited thereto. An electronic circuit (electronic device) other than the multiplexer may be hermetically sealed in the same manner.

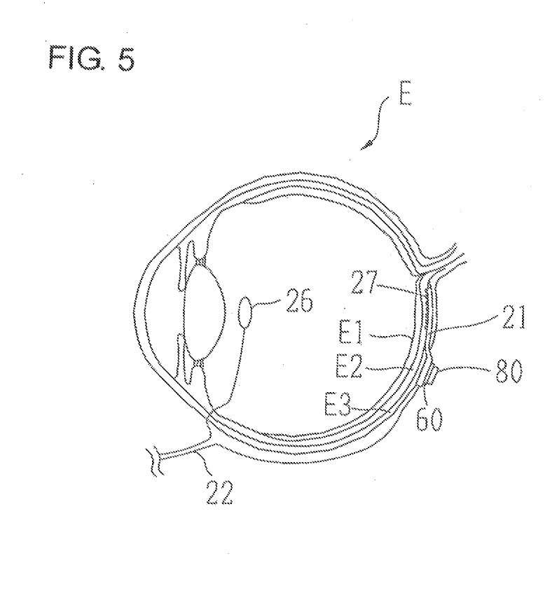

In the vision regeneration assisting apparatus according to the embodiment, the installation positions of the multiplexer 40, the electrodes 27, etc. on the substrate are considered in order to install the electrodes at positions capable of appropriately stimulating cells constituting the retina of a patient's eye. For example, as shown in FIG. 5, in the case where the electrodes 27 are provided on a choroid E2 so as to stimulate cells constituting the retina E1, as shown in FIG. 2B, a surface on the opposite side of the installation surface of the multiplexer 40 in the substrate 21 may be provided with a plurality of electrodes 27. In such an arrangement, the front end provided with the electrodes 27 of the substrate 21 is inserted into a sclera pocket manufactured by incising a part of a sclera E3 so as to stimulate cells constituting the retina E1. With such a configuration, since the multiplexer 40 does not come into contact with the retina E1 and the choroid E2 upon installing the internal body device 20 in the eyeball, the operation technique during the installation is comparatively simple.

A returning electrode 26 is disposed at a position close to the front eye portion at the center of the inside of the eye as shown in the drawing. Accordingly, the retina E1 is located between the electrode 27 and the counter electrode 26. The electric stimulus pulse signal current generated from the electrode 27 efficiently flows through the retina.

Meanwhile, the receiver 23 is provided at a predetermined position inside living tissue so as to receive a signal (the electric stimulus pulse data and the power) from the transmitter 14 provided in the external body device 10. For example, as shown in FIG. 1, the control unit 25 (only the receiver 23 is shown in the drawing) is buried in a position below the skin of a patient's temporal region, and the transmitter 14 is provided at a position facing the receiver 23 with the skin interposed therebetween. Since the receiver 23 is attached with a magnet as in the transmitter 14, when the transmitter 14 is located on the implanted receiver 23, the transmitter 14 and the receiver 23 attract each other by a magnetic force, so that the transmitter 14 is held in the temporal portion.

In addition, the cable 22 is extended below the skin from the control unit 25 embedded in the temporal region toward the patient's eye along the temporal region, and is inserted into an orbit through the inside of a patient's upper eyelid. The cable 22 inserted into the orbit passes the outside of the sclera E3 as shown in FIG. 5, and is connected to the multiplexer 40 provided in the substrate 21.

Incidentally, in the installation position of the internal body device 20 in the vision regeneration assisting apparatus, as shown in FIG. 5, a configuration is adopted in which the electrodes 27 are located at the sclera pocket manufactured in the sclera E3 so as to give an electric stimulus to cells from the sclera (choroid) to the retina E1, but the invention is not limited thereto. The electrodes may be provided at position where the cells constituting the retina of the patient's eye are appropriately stimulated. For example, a configuration may be adopted in which the internal body device is located in the inside (above or below the retina) of the patient's eye so that the front end portion of the substrate provided with the electrodes is fixed to a position below the retina (between the retina and the choroid) or a position above the retina. In addition, a configuration may be adopted in which the electrodes 27 are located in the sclera E3 so as to stimulate cells from the sclera (choroid) to the retina E1. Further, the substrate 21 may be fixed and held to the sclera E3 by using, for example, a tack or an adhesive having high biocompatibility.

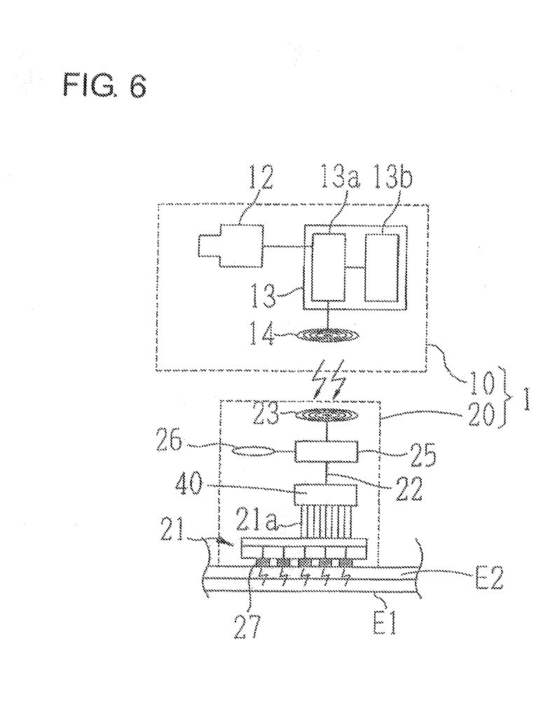

In the vision regeneration assisting apparatus with the above-described configuration, an operation for the vision regeneration will be described with reference to the block diagram of the control system shown in FIG. 6. Photographing data (image data) of the subject photographed by the photographing device 21 is transmitted to the pulse signal converter 13a. The pulse signal converter 13a converts the photographed subject into a signal (electric stimulus pulse data) within a predetermined bandwidth so as to be seen by the patient, and transmits the resultant from the transmitter 14 to the internal body device 20 as an electromagnetic wave.

At the same time, the pulse signal converter 13a converts the power supplied from the battery 13b into a signal (power) in a bandwidth different from the bandwidth of the above-described signal (electric stimulus pulse data), and transmits the resultant to the internal body device 20 together with the electric stimulus pulse data as an electromagnetic wave.

The internal body device 20 receives the electric stimulus pulse data and the power transmitted from the external body device 10 through the receiver 23, and transmits the resultant to the control unit 25. The control unit 25 extracts a signal within the bandwidth used by the electric stimulus pulse data from the received signal. The control unit 25 generates the electric stimulus pulse to be distributed to each of the electrodes 27 and the multiplexer control signal used for controlling the distribution of the electric stimulus pulse on the basis of the extracted electric stimulus pulse data, and transmits the electric stimulus pulse and the control signal to the multiplexer 40. The multiplexer 40 distributes the electric stimulus pulse to each of the plurality of electrodes 27 on the basis of the received control signal, and outputs the electric stimulus pulse from each of the electrodes 27. The cells constituting the retina are stimulated by the electric stimulus pulse output from each of the electrodes 27, and hence the patient's vision is regenerated.

Incidentally, in the above-described embodiment, the electrode is provided in the eyeball so as to stimulate cells constituting the retina, but the invention is not limited thereto. A configuration may be adopted in which the patient's vision is regenerated by stimulating tissues or cells constituting the vision. For example, a configuration may be adopted in which the electrode is provided in an optic nerve or a cerebral cortex.

Further, in the above-described embodiment, an example of the vision regeneration assisting apparatus is described in which a part of the internal body device as the functional device is provided in the patient's eyeball or head so as to regenerate the patient's vision, but the invention is not limited thereto. A configuration may be adopted in which the functional device is provided inside the living body. For example, a regeneration assisting apparatus for senses such as an acoustic sense or an olfactory sense may be adopted. In addition, an electric stimulus device may be adopted which is used for a treatment of a disease of a brain nervous system.

Furthermore, the unit electrically connected to the outside of the casing may not be provided. A configuration may be adopted in which a connection substrate is used for a connection of a connecting terminal (an connecting terminal inside a casing) of a circuit element (for example, a coil for transmitting and receiving information or receiving power). For example, a configuration may be adopted in which an electronic circuit used for a verification is hermetically sealed by using the technology of the invention, and is implanted into living tissue as a functional device unit.

What is claimed is:

1. A biological implantable functional device comprising:
a casing having a space for accommodating an electronic device and formed with an opening;
a bendable flexible wiring substrate in which a wiring is formed in a predetermined pattern so as to correspond to an device-side terminal of the electronic device;
a casing inner connecting terminal to be connected to an electric substrate provided outside the casing;
a bump to be electrically connected with the flexible wiring substrate and the casing inner connecting terminal; and
a cover for sealing the opening of the casing to hermetically seal the electronic device.

2. The biological implantable functional device according to claim 1, wherein the hermetically sealing is performed by ultrasonic joining or pressure joining.

3. The biological implantable functional device according to claim 1, wherein
the biological implantable functional device is used for a vision regeneration assisting apparatus, and
the casing inner connecting terminal is electrically connected to an exterior substrate which is covered by a biocompatible material, which is processed to have a thickness configured the exterior substrate to be bent, and includes a plurality of electrodes connected to a plurality of lead wires formed within the exterior substrate.

4. The biological implantable functional device according to claim 3, wherein the casing inner connecting terminal is electrically connected to the exterior substrate through a bump.

5. The biological implantable functional device according to claim 1, wherein
the flexible wiring substrate includes a base which is a film-shaped member formed from an insulating material, and
the wiring pattern is formed on a surface of the base.
